(19) United States
(12) Patent Application Publication
(10) Pub. No.: US 2004/0062741 A1
(43) Pub. Date: Apr. 1, 2004

(54) Aftershave composition containing phospholipid

(75) Inventors: Maureen Sullivan Morrissey, Belmont, MA (US); Stephen H. Thong, Needham, MA (US); Alexander Novikov, Framingham, MA (US)

Correspondence Address: PATENT AND TRADEMARK COUNSEL, THE GILLETTE COMPANY, 800 BOYLSTON STREET, BOSTON, MA, 02199, US

(73) Assignee: The Gillette Company

(21) Appl. No.: 10/259,249

(22) Filed: Sep. 27, 2002

(51) Int. Cl. A61K 7/15; A61K 9/127

(52) U.S. Cl. 424/73

(57) ABSTRACT

The present invention provides an aftershave composition comprising, by weight, 9% to 94%, preferably 15% to 85%, most preferably 25% to 55%, water, 5% to 90%, preferably 10% to 80%, most preferably 40% to 70%, ethanol, 0.25% to 6%, preferably 1% to 5%, fragrance, 0.25% to 5%, preferably 1% to 4%, surfactant to solubilize or emulsify the fragrance, and 0.05% to 1.5%, preferably 0.1% to 0.6%, phosphatidyl choline. Preferably, the composition will be a clear solution or microemulsion having a clarity better than 100 NTU and a viscosity less than 200 mPas. The foregoing composition provides skin moisturization and reduced irritation without other undesirable aesthetic attributes such as stickiness or oiliness. The present invention also embraces a method of reducing irritation of human skin caused by shaving comprising topically applying to human skin after shaving an aftershave composition as described above.

### BACKGROUND OF THE INVENTION

The present invention relates to an aftershave composition that provides skin moisturization and reduced irritation without an oily or sticky feeling. The aftershave composition includes a phospholipid in an aqueous-alcoholic base vehicle.

Numerous types of aftershave products are known. Many of these products have very high alcohol content (e.g. >50%), although some have low alcohol or no alcohol. Each type of formulation has its drawbacks. The high alcohol formulations tend to cause stinging and burning, while the low to no alcohol formulations tend to dry too slow and feel wet. None of these products provide skin moisturization or reduce irritation.

U.S. Pat. No. 4,784,849 describes oil-based shaving compositions that contain the phospholipid lecithin in an amount of 5% to 20% and other oils such as Vitamin E, wheat germ oil, etc. This type of composition would not make a suitable aftershave product and would be very oily feeling. U.S. Pat. No. 5,527,530 describes an alcoholic moisturizing aftershave lotion that contains an anti-inflammatory agent and an acyl lactylate moisturizing emollient. Of course, various skin care lotions containing lecithin are known, but these also would not make suitable aftershave products.

It is highly desirable to provide an aftershave composition which provides a combination of desirable aesthetic attributes without incurring further undesirable attributes. In particular, it is desirable to provide an aftershave composition that will provide skin moisturization and reduced irritation, without other undesirable aesthetic attributes such as stickiness or oiliness.

### SUMMARY OF THE INVENTION

An aftershave composition in accordance with the present invention comprises, by weight, 9% to 94%, preferably 15% to 85%, most preferably 25% to 55%, water, 5% to 90%, preferably 10% to 80%, most preferably 40% to 70%, ethanol, 0.25% to 6%, preferably 1% to 5%, fragrance, 0.25% to 5%, preferably 1% to 4%, surfactant to solubilize or emulsify the fragrance, and 0.05% to 1.5%, preferably 0.1% to 0.6%, phosphatidylcholine. Preferably, the composition will be a clear solution or microemulsion having a clarity better than 100 NTU (i.e., 0-100 NTU), most preferably better than 50 NTU (i.e., 0-50 NTU). In addition, the composition ideally will have a viscosity less than 200 mPas, most preferably less than 100 mPas. The foregoing composition provides skin moisturization and reduced irritation without other undesirable aesthetic attributes such as stickiness or oiliness. The present invention also embraces a method of reducing irritation of human skin caused by shaving comprising topically applying to human skin after shaving an aftershave composition as described above.

### DETAILED DESCRIPTION OF THE INVENTION

The aftershave composition of the present invention is primarily an aqueous-alcoholic solution or microemulsion containing a fragrance. The composition may comprise 5% to 90%, preferably 10% to 80%, most preferably 40% to 70%, ethanol. Any fragrance or perfume may be employed so long as it may be effectively solubilized or emulsified in the composition. The amount of fragrance will typically fall within the range of about 0.25% to 6%, preferably 1% to 5%. A surfactant is added to the composition in order to solubilize or emulsify the fragrance. The surfactant is typically a polyethoxylated and/or polypropoxylated surfactant such as, for example, PPG-26-Buteth-26 and PEG-40 Hydrogenated Castor Oil. Naturally, of course, other known fragrance solubilizing surfactants may be employed for this purpose. See, for example, U.S. Pat. No. 4,279,891, U.S. Pat. No. 5,527,530, EP 261,351, EP 571,677, and WO 96/03963, the disclosures of which are incorporated herein by reference. The amount of surfactant will typically fall within the range of about 0.25% to 5%, preferably 1% to 4%.

The aftershave composition will additionally comprise 0.05% to 1.5%, preferably 0.1% to 0.6%, phosphatidylcholine as a moisturizing agent. The phosphatidylcholine should be substantially free of other phospholipids (i.e., it should be at least about 90% purity) and should be essentially neutral (i.e., no ionic charge). Preferably, the phosphatidylcholine is derived from a plant, most preferably soy bean.

The aftershave composition of the present invention may also include other cosmetic ingredients to provide desirable skin benefits or aesthetic attributes. Such ingredients may include emollients, cooling agents, preservatives, viscosity modifiers, coloring agents, moisturizing agents, skin conditioning agents, medicinal agents, etc.

The present invention may be further illustrated by the following examples in which the parts and percentages are by weight.

### EXAMPLES 1 TO 6

Aftershave compositions are prepared having the ingredients and the amounts set out below. Each of these compositions is prepared by dissolving the phosphatidylcholine in ethanol, adding the fragrance and surfactant, then mixing with water to provide a clear solution or microemulsion.

| | Weight Percent | | | | | |
|---|---|---|---|---|---|---|
| Ingredient | Ex. 1 | Ex. 2 | Ex. 3 | Ex. 4 | Ex. 5 | Ex. 6 |
| Water | 29.9 | 54.3 | 39.3 | 75.4 | 89.7 | 65.4 |
| Ethanol | 65.0 | 40.0 | 55.0 | 20.0 | 5.0 | 30.0 |
| Fragrance | 4.5 | 3.0 | 4.0 | 2.5 | 2.5 | 2.5 |
| Surfactant¹ | 0.5 | 2.5 | 1.5 | 2.0 | 2.5 | 2.0 |
| Phosphatidylcholine² | 0.1 | 0.2 | 0.2 | 0.1 | 0.3 | 0.1 |

¹PEG-40 Hydrogenated Castor Oil or PPG-26-Buteth-26 or a mixture of these surfactants
²Phospholipon 90 G (American Lecithin Co.; 93 ± 3% purity; derived from soy beans)

What is claimed is:

1. An aftershave composition comprising, by weight, 9% to 94% water, 5% to 90% ethanol, 0.25% to 6% fragrance, 0.25% to 5% surfactant to solubilize or emulsify the fragrance, and 0.05% to 1.5% phosphatidylcholine.

2. The composition of claim 1 comprising 15% to 85% water and 10% to 80% ethanol.

3. The composition of claim 1 comprising 25% to 55% water and 40% to 70% ethanol.

4. The composition of claim 1, 2 or 3 comprising 0.1% to 0.6% phosphatidylcholine.

5. The composition of claim 4 wherein the phosphatidylcholine is substantially free of other phospholipids.

6. The composition of claim 1 consisting essentially of 15% to 85% water, 10% to 80% ethanol, 0.25% to 6% fragrance, 0.25% to 5%, surfactant, and 0.05% to 1.5% phosphatidylcholine substantially free of other phospholipids.

7. The composition of claim 1 consisting essentially of 25% to 55% water, 40% to 70% ethanol, 0.25% to 6% fragrance, 0.25% to 5%, surfactant, and 0.05% to 1.5% phosphatidylcholine substantially free of other phospholipids.

8. The composition of claim 1, 2 or 3 in the form of a clear solution or microemulsion having a clarity better than 100 NTU.

9. The composition of claim 8 having a viscosity less than 200 mPas.

10. A method of reducing irritation of human skin caused by shaving comprising topically applying to human skin after shaving an aftershave composition comprising, by weight, 9% to 94% water, 5% to 90% ethanol, 0.25% to 6% fragrance, 0.25% to 5% surfactant to solubilize or emulsify the fragrance, and 0.05% to 1.5% phosphatidylcholine.

11. The method of claim 10 wherein said aftershave composition comprises 15% to 85% water and 10% to 80% ethanol.

12. The method of claim 11 wherein said aftershave composition comprises 25% to 55% water and 40% to 70% ethanol.

13. The method of claim 10, 11 or 12 wherein said aftershave composition comprises 0.1% to 0.6% phosphatidylcholine.

14. The method of claim 13 wherein the phosphatidylcholine is substantially free of other phospholipids.

15. The method of claim 10 wherein said aftershave composition consists essentially of 15% to 85% water, 10% to 80% ethanol, 0.25% to 6% fragrance, 0.25% to 5%, surfactant, and 0.05% to 1.5% phosphatidylcholine substantially free of other phospholipids.
